(19) United States
(12) Patent Application Publication
(10) Pub. No.: US 2008/0200859 A1
(43) Pub. Date: Aug. 21, 2008

(54) Apheresis systems & methods

(76) Inventors: Mehdi Hatamian, Coto De Caza, CA (US); Matthew Douglas McCawley, San Clemente, CA (US); Toshi Ghalebi, Rancho Santa Margarita, CA (US); Matin Ebneshahrashoob, Huntington Beach, CA (US)

Correspondence Address: STETINA BRUNDA GARRED & BRUCKER, 75 ENTERPRISE, SUITE 250, ALISO VIEJO, CA, 92656, US

(21) Appl. No.: 11/706,633

(22) Filed: Feb. 15, 2007

(51) Int. Cl. A61M 1/38

(52) U.S. Cl. 604/6.02

(57) ABSTRACT

A system for separating fluid components comprising a sealless rotating drum, a rotatable disposable centrifuge cartridge within the drum, a multi-lumen tube fluidly connectable to the cartridge and to an external fluid source, and a control system capable of independently controlling the flow rate within each lumen of the multi-lumen tube. This system may also be used in apheresis procedures for separating blood components from whole blood. The multi-lumen tube may have a ribbon section that unidirectionally fits within a peristaltic block of rollers, wherein each lumen's flow rate may be individually controlled. The system may further include a camera unit operable to observe a separation boundary within the centrifuge cartridge and accordingly adjust the rotation speed to increase or decrease the amount of separation.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Field of the Invention

The present invention relates to systems and methods for processing fluids and fluid components. More particularly, the present invention relates to an apheresis system for separating whole blood from a donor into at least two components.

2. Description of Related Art

Continuous flow fluid separation is useful in many chemical, medical, research, and industrial contexts. Many times, fluids mix with other fluids and it is desired to reverse that process and separate those fluids, sorting the fluid subcomponents according to density and/or molecular weight. In some cases, particles are present in solution and these particles need to be precipitated out of or removed from the solution.

Blood apheresis is one common medical use of continuous fluid separation. Apheresis has many clinical uses, including multiple therapies that involve removing blood from a patient's body, separating the blood into components, altering one of the components, and putting some mixture or selection from the removed and/or altered fluid back into the patient's body. Some exemplary therapeutic apheresis procedures include: therapeutic plasma exchange (TPE), a procedure by which cell-free plasma is removed and replaced with colloid/saline solution; cytoreduction, a process by which platelets and white blood cells are removed; photopheresis, a procedure by which mononuclear cells collected by therapeutic apheresis are exposed to ultraviolet-A light and psoralen, and reinfused into the patient; and selective adsorption, a process by which plasma is adsorbed on a column and returned to the patient.

Additionally, efficient apheresis can be used to provide a more efficient and less uncomfortable experience for those who wish to donate blood, in addition to helping make donated blood safer for clinical use. For example, if only a portion of the blood is in demand, that portion can be separated and the remaining portions can flow back in to the donor. Many other applications exist. For example, apheresis can be used to test athletes for doping violations without excess blood removal, and those who have had a drug overdose can be treated by detoxifying their blood with apheresis techniques.

In a basic apheresis procedure, blood is withdrawn from a donor through a needle inserted into the vein of a donor. The needle is attached to one end of a plastic tube which provides a flow path for the blood. The other end of the tube leads to a separator, such as a centrifuge, for separating the blood into its components. Flow-through centrifuges which allow for the continuous inflow and outflow of materials to and from the centrifuge are well known within the art. For example, U.S. Pat. No. 4,425,112, which is hereby incorporated by reference in its entirety, describes a sealless centrifuge that allows the continuous transfer of material to and from a centrifuge bowl via tubes which are directly connected to the bowl from the outside of the centrifuge without the use of rotating seals. This result is achieved by threading a multi-lumen tube through the top housing of a centrifuge drum, the multi-lumen tube enters the housing inline with the driveshaft of the centrifuge. The multi-lumen tube extends radially outward from the axis of rotation of the drive shaft to a hollow outer shaft located within a first circular plate. The multi-lumen tube extends downwardly through the hollow outer shaft, and then extends radially inward to a hollow center shaft within the first circular plate. The multi-lumen tube then extends upwardly though the hollow center shaft of the first plate and attaches in fluid communication to the centrifuge bowl located above the first circular plate. The first circular plate is driven by the drive shaft at a predetermined angular velocity ω. By utilizing pulleys and a 1:1 gear ratio, the centrifuge bowl rotates at an angular velocity of 2ω. Finally, the hollow outer shaft of the first plate is caused to rotate about its own axis at an angular velocity of −ω, thereby causing the multi-lumen tube to remain untwisted while also allowing fluid communication into and out of the centrifuge bowl without any rotating seals.

Automated apheresis systems are now routinely used which utilize disposable, pre-sterilized fluid circuits through which the blood flows. The fluid circuits are mounted on reusable machines that may have pumps, valves, sensors, and the like. These automated systems further include an internal computer and associated software programs which control many of the processing functions. The fluid circuits of these automated systems, however, typically have complex routing procedures that require skill and training by the apheresis operator in order to assemble the system. For example, U.S. Pat. No. 6,706,008 B2, which is herein incorporated by reference in its entirety, illustrates and describes an automated apheresis system. Although this system is simpler and easier to use than previous apheresis systems, it still includes complex routings of tubes through peristaltic pump rotors which can be difficult to properly assemble and/or can be assembled improperly.

Additionally, existing apheresis processes can have negative effects on patients. For example, many apheresis and similar processes draw blood in sudden, relatively large doses from the patient, causing trauma, nausea, or other harmful side effects. These large draws are often repeated in order to obtain enough blood for the desired medical test or therapy, but the effect of repeated heavy draws of blood from a patient can be harmful. Furthermore, existing methods can be inefficient and can cause inconvenient delays in the time it takes for blood to separate or travel through the apheresis machine. Moreover, many existing apheresis systems are expensive and difficult to assemble by the apheresis operator. Therefore, a need exists for improved systems and methods for separating fluids. In particular, a need exists for improved systems and methods for efficiently separating blood constituents in a continuous flow apheresis device, and for apheresis devices that are easier to assemble and operate.

### BRIEF SUMMARY

Certain embodiments of the present invention include a fluid component separation system having a sealless rotating drum, a disposable centrifuge cartridge, a multi-lumen tube, and a control system. The sealless rotating drum includes a first independently rotatable disc as well as a second independently rotatable disc; the first disc being positioned above the second disc within the drum. The disposable centrifuge cartridge is capable of being temporarily attached to the first disc during the fluid separation process. The multi-lumen tube can attach to the centrifuge cartridge at one end and is capable of attaching at least to a fluid source located outside of the drum at the other end, thereby being able to transmit fluid to and from the centrifuge cartridge. The control system is capable of independently controlling the flow rate of liquid within each lumen of the multi-lumen tube.

Additionally, the disposable centrifuge cartridge may include stacked separation levels for separating fluid components by their density. The multi-lumen tube may include any number of lumens, but in one embodiment of the invention it is envisioned that the tube will have six lumens.

Furthermore, the control system may be a peristaltic block including rows of independently controllable caterpillar rollers. The number of roller rows may be made to correspond to the number of lumens within the multi-lumen tube. In particular, the multi-lumen tube may further include a ribbon section, wherein the lumens are aligned in a plane so that each lumen may be positioned in contact with an independently controlled caterpillar roller row within the peristaltic block. Although, the number of roller rows and lumens may correspond, it is also envisioned that a peristaltic block may have a greater number of rollers than is present in the multi-lumen tube used with the block.

Additionally, the ribbon section of the multi-lumen tube may be bounded on each side by fittings, wherein each fitting is shaped differently than the other. Optionally, at least one of these fittings may be asymmetric in form. The peristaltic block will be designed to include compatible recesses, which are capable of receiving the ribbon section fittings. By having dissimilar fittings on each end of the ribbon section, including at least one which may be asymmetric in form, improper operator installation of the multi-lumen tube in the peristaltic block is minimized, since the tube will only fit into the block in the intended direction.

Another embodiment of the present invention includes an apheresis system useful for separating whole blood from a patient into at least two components. The apheresis system includes a sealless rotating drum, a disposable centrifuge cartridge, a multi-lumen tube, and a control system. The sealless rotating drum includes a first independently rotatable disc as well as a second independently rotatable disc; the first disc being positioned above the second disc within the drum. The disposable centrifuge cartridge is capable of being temporarily attached to the first disc during the blood component separation process. The multi-lumen tube can attach to the centrifuge cartridge at one end and is capable of attaching at least to a blood source located outside of the drum at the other end, thereby being able to transmit blood and blood components to and from the centrifuge cartridge. The control system is capable of independently controlling the flow rate of liquid within each lumen of the multi-lumen tube.

The disposable centrifuge cartridge may include stacked separation levels for separating blood components by their density. In particular, the centrifuge cartridge may include three separation levels stacked on top of each other. When the centrifuge cartridge includes three separation levels, the upper separation level may separate out red blood cells from the remainder of the blood before routing the remaining blood components to the mid separation level. The mid separation level may then separate out component poor plasma before routing the remaining component rich plasma to the lower separation level. The lower separation level may then separate white blood cells from platelets.

Although the multi-lumen tube may contain as many lumens as is necessary for a given procedure, it is envisioned that in one embodiment the multi-lumen tube may include six lumens. In particular, one lumen may convey blood or blood components to and from the centrifuge cartridge and to and from a blood source. This blood source may be a donor giving blood during the apheresis process. The blood source also may be a supply of blood previously drawn from a donor prior to the apheresis process.

After separation, the blood components may be routed out of the centrifuge cartridge by dedicated lumens within the multi-lumen tube. These blood components may be routed through their individual lumens in numerous ways, including, but not limited to, routing them back to the patient, routing them to a collection bag for storage, or routing them to another system for further processing.

The control system of the apheresis unit may be a peristaltic block made up of rows of independently controllable caterpillar rollers. The number of roller rows may be made to correspond to the number of lumens within the multi-lumen tube. In addition, the multi-lumen tube may further include a ribbon section. In this ribbon section, the lumens are aligned in a plane so that each lumen may be positioned in contact with an independently controlled caterpillar roller row of the peristaltic block. Although, the number of roller rows and lumens may correspond, it is also envisioned that a peristaltic block may have a greater number of rollers than is present in the multi-lumen tube used with the block.

Additionally, the ribbon section of the multi-lumen tube may be bounded on each side by fittings, wherein each fitting is shaped differently than the other. Optionally, at least one of these fittings may be asymmetric in form. The peristaltic block will be designed to include compatible recesses, which are capable of receiving the ribbon section fittings. By having dissimilar fittings on each end of the ribbon section, including at least one which may be asymmetric in form, improper operator installation of the multi-lumen tube in the peristaltic block is minimized, since the tube will only fit into the block in the intended direction.

The apheresis system of the present invention may further include a camera unit mounted above the centrifuge cartridge. In this case, the top of the centrifuge cartridge may be transparent so that the contents of the centrifuge cartridge may be viewed by the camera unit. The camera unit thus may be able to differentiate a separation boundary within the centrifuge cartridge. This separation boundary may mark the line between separation of red blood cells, and the remaining blood components.

The apheresis system of the present invention may further include a microcontroller. This microcontroller may communicate with the camera unit in order to receive separation boundary information from the camera unit. The microcontroller may then adjust the angular velocity of the centrifuge cartridge accordingly based on the separation boundary information. In particular, if the separation boundary is below a desired limit, the microcontroller may increase the angular velocity of the centrifuge cartridge in order to increase the level of separation.

Another embodiment of the present invention includes a method for separating whole blood obtained from a donor into at least two components. In order to effectuate this method, one supplies a source of whole blood to a sealless rotating drum. The blood enters the drum via a dedicated lumen within a multi-lumen tube. The whole blood is then separated into at least two components based on the density of the various components. This separation is achieved via centrifugal forces placed on the components within a rotating centrifuge cartridge located within the rotating drum. Finally, the blood components are directed out of the rotating drum via separate dedicated lumens with the multi-lumen tube.

In this method, the flow rate of blood and blood components within each lumen of the multi-lumen tube may be controlled independently. Also, improved separation of blood components may be achieved in this method in numerous varied ways. For example, separation may be improved by increasing the angular velocity of the rotating centrifuge cartridge. Separation of blood components may also be improved by increasing the flow rate of an individual blood component leaving the centrifuge cartridge. Another manner for improving blood component separation is by decreasing the flow rate of an individual blood component. Additionally, one may combine the above improvements. For example, blood component separation can be improved by increasing the flow rate of one blood component while simultaneously decreasing the flow rate of a different blood component.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of the sealless rotating drum present in certain embodiments of the invention;

FIG. 2 is a side, cut-out view of the sealless rotating drum of FIG. 1;

FIG. 3 is a perspective view of the multi-lumen tubing present in certain embodiments of the invention;

FIG. 4 is a perspective view of the flow control system present in certain embodiments of the present invention;

FIG. 5 is a top view of the separation cartridge present in certain embodiments of the present invention;

FIG. 6A is a perspective view of the camera control system in relation to the separation cartridge of FIG. 5; and

FIG. 6B is a close-up view of the camera view region of the cartridge, as shown in FIG. 6A.

### DETAILED DESCRIPTION

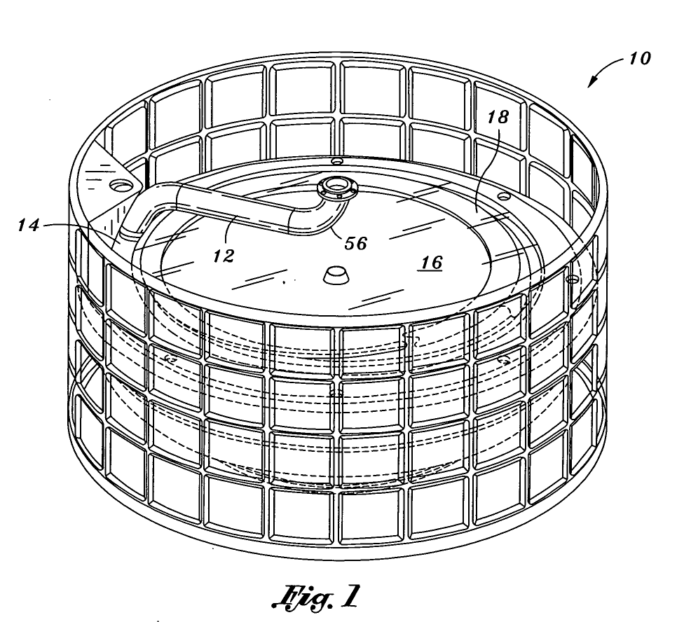

FIG. 1 illustrates a sealless rotating drum 10 present in certain embodiments of the present invention. The drum 10 may include a tube housing 56 capable of receiving a multi-lumen tube 12. The multi-lumen tube 12 may be routed through the entire length of the tube housing 56 so that both ends of the multi-lumen tube 12 extend beyond the length of the tube housing 56. The multi-lumen tube 12 may enter the drum 10 from the outside of the unit, via the tube housing 56, inline with the axis of rotation of the drum 10. The drum 10 may further include an independently rotatable central disc 14, an independently rotatable upper disc 16, and a disposable cartridge 18.

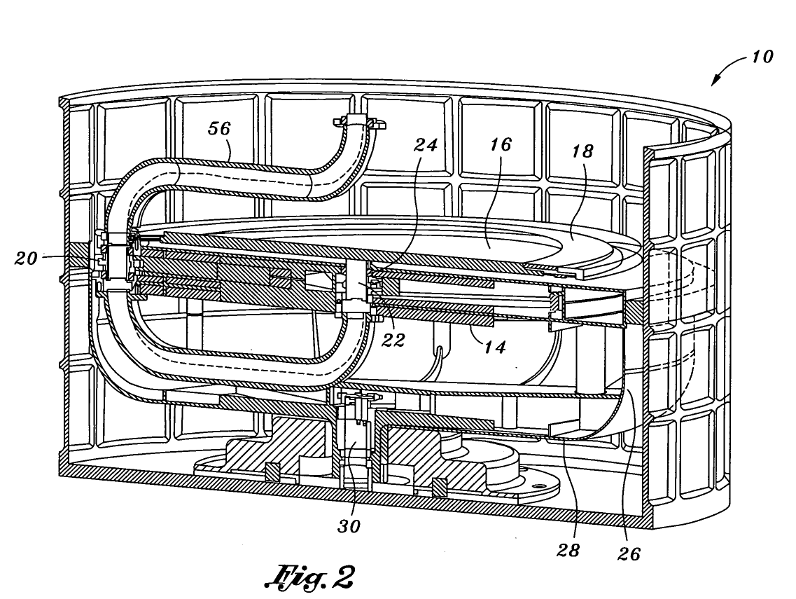

As can best be seen in FIG. 2, the tube housing 56 enters the drum 10 inline with the axis of rotation of the drum 10. The tube housing 56 then extends radially outward before entering and proceeding downward through a hollow channel 20 located on the periphery of a central disc 14. The tube housing 56 then proceeds radially inward below the central disc 14 before entering and proceeding upward through a centrally located hollow channel 22 in the central disc 14. The tube housing 56 may also proceed through a centrally located hollow channel 24 in the upper disc 16. The tube 12, routed through the tube housing 56, terminates within the drum 10 by attaching to and maintaining fluid connection with the disposable cartridge 18 attached to the upper disc 16. The tube housing 56 may be a rigid structure that surrounds the multi-lumen tube 12 within the drum 10. Since the multi-lumen tube 12 may be routed through the tube housing 56 from the entry point of the drum 10 to the upper disc 16, all references herein to the multi-lumen tube 12 when inside the drum 10 may also refer to the tube housing 56 enclosing the multi-lumen tube 12. It is contemplated that the disposable cartridge 18 may be attached to the upper disc 16 in any number of ways known within the art for easily and quickly securing one article to another. For example, one way of attaching the disposable cartridge 18 to the upper disc 16 may be by way of a snap-fit mechanism that may include tabs on the cartridge 18 that fit within and releasably engage holes in the upper disc 16, or also by a snap-fit mechanism whereby the cartridge 18 includes holes that may be releasably engaged by tabs formed on the upper disc 16.

The central disc 14 is attached by a sidewall 26 to a lower disc 28, which is likewise attached to a drive unit 30. The drive unit 30 causes the lower disc 28, and consequently the central disc 14, to rotate at a predetermined angular velocity ω. By virtue of the sidewall 26 attaching the central disc 14 to the lower disc 28, the central disc 14 may be rotated at angular velocity ω by the drive unit 30 without the need for a center shaft support system. The rotation of the central disc 14 causes the peripheral hollow channel 20, as well as the multi-lumen tube 12 when inserted therein, to move in an orbital path around the interior periphery of the drum 10. By conventional methods well known within the art, the peripheral hollow channel 20, and consequently the multi-lumen tube 12 inserted therein, is caused to rotate itself at an angular velocity of −ω thereby preventing twisting of the tube 12 between the entry point of the tube 12 within the drum 10 from the exterior and the peripheral hollow channel 20. By these same conventional methods, the upper disc 16, and consequently the disposable cartridge 18 attached to the upper disc 16, is caused to rotate at an angular velocity 2ω. This rotation of twice the speed prevents twisting of the tube 12 between the peripheral hollow channel 20 and the connection point of the tube 12 at the disposable cartridge 18. Exemplary methods for creating angular velocities as described above is discussed in U.S. Pat. No. 4,425,112, the entire contents of which are incorporated herein by reference.

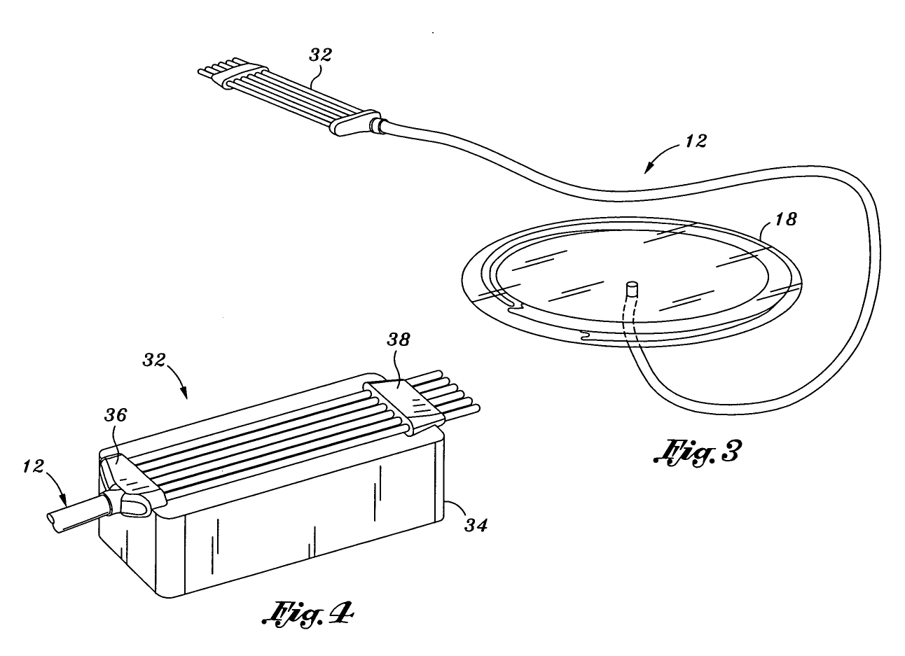

FIG. 3 illustrates the disposable multi-lumen tubing 12 present in certain embodiments of the present invention. One end of the tube 12 is shown in fluid connection with the disposable cartridge 18 of the present invention. Shown at the other end of the tube 12, is the ribbon component 32 of the multi-lumen tube 12. As can best be seen in FIG. 4, the multiple lumens are separated out at the ribbon component 32 into an inline section wherein all of the lumens lie within a plane. The ribbon component 32 of the tube 12 is attached to a peristaltic block 34 for control of the flow of liquids into and out of the drum 10. Each lumen of the ribbon component 32 is located above its own caterpillar roller in the peristaltic block 34. By having individual caterpillar rollers for each lumen of the tube 12, flow rates for each lumen may be controlled individually. Furthermore, individual flow rates are achieved without requiring complex routing or circuits as was needed in the prior art. The individual lumens of the ribbon component 32 are collected together in a first fitting 36 and then incorporated into a single tube to facilitate operator loading and unloading of the tube 12 within the drum 10. On the other end of the ribbon component 32, the individual lumens are connected by a second fitting 38 in order to maintain a cohesive unit within the ribbon component 32 area, however, the lumens may then be separated after the second fitting 38 in order to be routed to the desired locations, e.g., to the patient, collection bags, component bags and the like. To further facilitate the proper loading of the tubing 12 on the peristaltic block 34, the first fitting 36 and the second fitting 38 may be made in the form of dissimilar shapes, with corresponding recesses present in the peristaltic block 34 so that the ribbon component 32 may only be placed on the peristaltic block in the proper unidirectional fashion. Additionally, in order to ensure that the ribbon component 32 is attached to the peristaltic block 34 in the proper orientation one of the fittings, for example the first fitting 36, may be formed in an asymmetric shape with a corresponding asymmetric recess present in the peristaltic block 34 in order to allow the ribbon component 32 to be placed on the peristaltic block 34 only in the proper orientation.

Although the multi-lumen tube 12 is shown in FIGS. 3 and 4 with six lumens, it is contemplated that a tube 12 may be used with any number of lumens. Furthermore, it is not necessary to use a different peristaltic block 34 for different numbered lumen tubes 12. For example, multi-lumen tubes 12 of differing lumen numbers may be adapted to fit on a single peristaltic block capable of accepting a large lumen number tube 12. These smaller number lumen tubes 12 may have first and second fittings 36, 38 that match the corresponding recesses present in the peristaltic block 34, even if the fittings 36, 38 are larger than necessary for the smaller number lumen tubes 12. In this example, the fittings 36, 38 will have blank spots with no lumens present, in order to fit on the peristaltic block 34. Operation of the peristaltic block 34 may be altered accordingly so that the rollers under the blank spots will be non-operational in these embodiments.

The peristaltic block 34 may be controlled by a computer program which regulates the speed of each independent roller as is necessary. By adjusting the speed of each roller, flow into and out of the system may likewise be adjusted. Furthermore, as will be discussed in greater detail below, adjusting the flow rate of certain components may allow for changes in separation of the components.

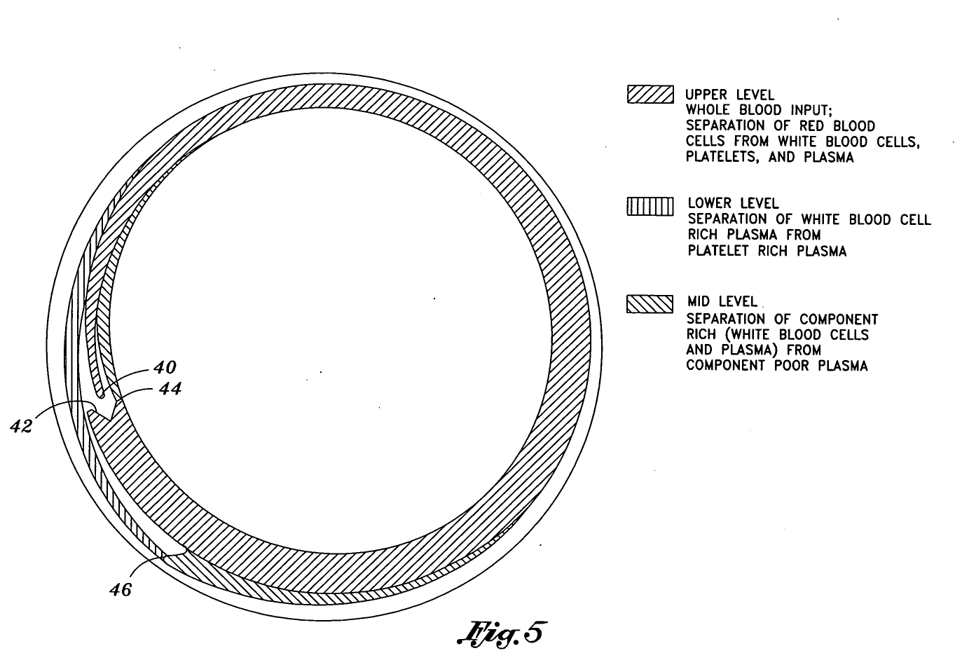

FIG. 5 illustrates one particular configuration of the disposable cartridge 18 used with the present invention. Although this example is exemplary of a flow paths envisioned for the disposable cartridge 18, it is contemplated that numerous other flow path configurations may be utilized with the present invention. In this embodiment, the disposable cartridge 18 comprises three stacked separation levels. Whole blood from the patient is directed from one lumen of the tube 12 into a whole blood input 40 located in the upper level of the disposable cartridge. Each level is radially disposed around the outer edge of the cartridge 18 and terminates in inner and outer exits. The blood placed within the cartridge will be separated due to the forces placed on the components from the centrifugation, so that the denser components will migrate outwards and the less dense components will remain on the inner section of the levels. The upper level terminates in an outer red blood cell (RBC) exit 42 and an inner first step exit 44. Since red blood cells are the densest component of blood they will migrate outwards in the upper level of the cartridge 18 and be directed through the RBC exit 42 to a particular lumen within the tube 12. This RBC lumen may either return to the patient, be directed to a collection bag, or to some other processing apparatus. The less dense blood components will remain on the inner portion of the upper level and will be directed through the first step exit 44 to the mid level of the cartridge 18. It is envisioned that within the mid level of the cartridge 18, component poor plasma will be separated from component rich plasma. In particular, plasma containing white blood cells (WBC) and platelets will be denser and therefore migrate outwards in comparison to blood plasma lacking WBCs and platelets. The component rich plasma will therefore be directed through an outer second step exit 46 to the lower level of the cartridge 18 for further separation. The component poor plasma will likewise be directed though a plasma exit (not shown) to a plasma lumen within the tube 12. Again, similar to the RBC lumen, the plasma lumen may be directed back to the patient, to a collection bag, or to some other processing apparatus. The component rich plasma will be separated in the lower level into WBCs and platelets. In particular, the denser WBCs will migrate outwards and exit the lower level through a WBC exit (not shown) and the comparatively less dense platelets will remain on the inside of the level and exit through a platelet exit (not shown). The WBCs and platelets will be directed to WBC and platelet lumens, respectively, within the tube 12, and again may be directed back to the patient, to collection bags, or to other processing apparatuses.

The disposable cartridge 18 illustrated in FIG. 5, and described above, is just one particular embodiment of a disposable cartridge 18 that may be used with the present invention, and it is to be understood that alterations of the flow paths within the cartridge 18 are within the spirit and scope of the present invention. Additionally, as discussed above, the disposable cartridge 18 may be adapted to easily snap into place on the upper disc 16 in order to facilitate easy loading for the operator. Although an easy fit snap-on connection is one method of attaching the disposable cartridge 18 to the upper disc 16, any other means of attaching two components may be used, for example, pins, screws, etc.

Since the flow rates through each lumen within the tube 12 may be independently controlled via the peristaltic block 34, it is additionally envisioned that separation of the components may be fine tuned by changing particular flow rates in response to the achieved separation. For example, the components exiting the drum 10 may be analyzed for concentration and particular flow rates may be accordingly adjusted in order to achieve a more desired separation of components. By increasing the exit flow rate of a component within a particular level, more of the blood will be drawn through that particular exit resulting in a more dilute separation. Thus, for example, if the concentration of RBCs leaving through the RBC lumen is lower than would be expected, the flow rate of the RBC lumen can be increased in order to draw more of the fluid through that lumen, thereby increasing the RBC content. In contrast, if the RBC lumen is being “tainted” with non-RBC components, the RBC lumen flow rate can be decreased in order to allow for greater separation of the components before exiting the upper level. Although this was explained using RBCs, it is envisioned that any flow rate could be adjusted in this manner leading to improved separation of any component.

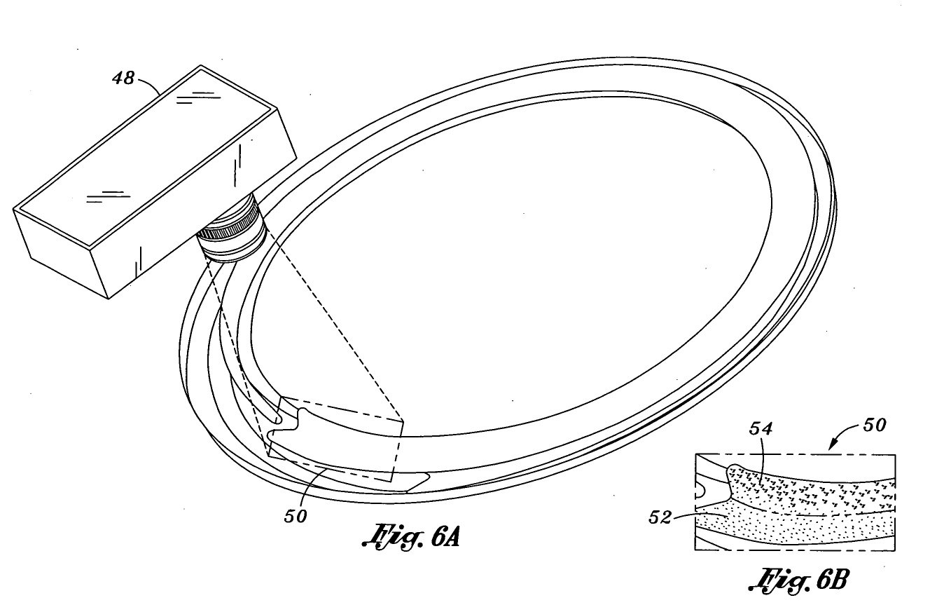

FIGS. 6A and 6B illustrate another aspect of the present invention, namely a vision control system utilized in order to help effectuate the desired separation of components within the disposable cartridge 18. The vision control system comprises a camera 48 mounted above the disposable cartridge 18. In this embodiment, the top of the upper level of the cartridge 18 is transparent allowing for visualization of the fluid within. The camera 48 views the separation boundary 50 located within the upper level of the disposable cartridge 18. In particular, as can be seen in detail in FIG. 6B, the camera 48 is able to view the contrast present in the upper level between the RBCs 52 and the other blood components 54. If the separation is not discrete enough, the camera 48 may communicate with a microcontroller which provides servo control of the centrifuge rotor thereby causing an increase in the angular velocity of the disposable cartridge 18 and thereby leading to an increase in separation of the blood components. The camera and/or microcontroller may also be in communication with the peristaltic block 34 and thereby adjust the flow rate of individual components as may be necessary. For example, if the separation contrast is not determined to be discrete enough, the exit flow rate of RBCs may be decreased thereby allowing for more time within the upper level of the cartridge, in order to gain a higher level of separation. In this embodiment, the camera 48 is only able to visualize the component boundary present in the upper level of the cartridge 18. Any further separations conducted in the mid or lower levels of the cartridge are dictated by the channeling present within the cartridge and/or the adjustment to flow rates as determined by the peristaltic block 34 and any external monitoring of component concentrations.

The above description is given by way of example, and not limitation. Given the above disclosure, one skilled in the art could devise variations that are within the scope and spirit of the invention disclosed herein, including various ways of designing and operating the sealless rotating drum. In particular, it is contemplated that the apheresis system of the present invention may be controlled by a microcomputer, wherein the operator inputs the desired apheresis conditions and the microcomputer controls the operating conditions of the system, including but not limited to the rotation speed of the drums, the flow rates of each individual lumen in the multi-lumen tube, and the degree of separation contrast in the upper level. Additionally, the configuration of the disposable cartridge may be altered in numerous ways, for example, any number of stacked levels may be present in the cartridge depending on the desired separation requirements. Also, the multi-lumen tube may contain any number of lumens as may be needed for particular applications. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A fluid component separation system comprising:
a) a sealless rotating drum comprising a first independently rotatable disc and a second independently rotatable disc, wherein said first disc is positioned above said second disc;
b) a disposable centrifuge cartridge, wherein said cartridge is releasably attachable to the first disc;
c) a multi-lumen tube fluidly connectable to said cartridge at a first end and to at least a fluid source located external to the sealless rotating drum at a second end; and
d) a control system for independently controlling the flow rate within each lumen of the multi-lumen tube.

2. The fluid component separation system of claim 1, wherein the disposable centrifuge cartridge comprises stacked separation levels for separating fluid components by their density.

3. The fluid component separation system of claim 1, wherein the multi-lumen tube comprises six lumens.

4. The fluid component separation system of claim 1, wherein the control system comprises a peristaltic block of independently controllable caterpillar rollers.

5. The fluid component separation system of claim 4, wherein the multi-lumen tube further includes a ribbon section, wherein the lumens are aligned in a plane so that each lumen may be positioned in contact with an independently controlled caterpillar roller of said peristaltic block.

6. The fluid component separation system of claim 5, wherein said ribbon section is bounded on each side by fittings of differing configurations.

7. The fluid component separation system of claim 6, wherein at least one of said fittings is asymmetric in form.

8. The fluid component separation system of claim 6, wherein the peristaltic block includes compatible recesses capable of receiving said ribbon section fittings.

9. An apheresis system for separating whole blood from a patient into at least two components, said apheresis system comprising:
a) a sealless rotating drum comprising a first independently rotatable disc and a second independently rotatable disc, wherein said first disc is positioned above said second disc;
b) a disposable centrifuge cartridge, wherein said cartridge is releasably attachable to the first disc;
c) a multi-lumen tube fluidly connectable to said cartridge at a first end and to at least a blood source located external to the sealless rotating drum at a second end; and
d) a control system for independently controlling the flow rate within each lumen of the multi-lumen tube.

10. The apheresis system of claim 9, wherein the disposable centrifuge cartridge comprises stacked separation levels for separating fluid components by their density.

11. The apheresis system of claim 10, wherein the centrifuge cartridge comprises three stacked separation levels.

12. The apheresis system of claim 11, wherein an upper separation level separates red blood cells from the remainder of the blood components.

13. The apheresis system of claim 11, wherein a mid separation level separates component poor plasma from component rich plasma.

14. The apheresis system of claim 11, wherein a lower separation level separates white blood cells from platelets.

15. The apheresis system of claim 9, wherein the multi-lumen tube comprises six lumens.

16. The apheresis system of claim 15, wherein one lumen conveys blood or blood components to and from the centrifuge cartridge and to and from a blood source.

17. The apheresis system of claim 15, wherein at least one lumen conveys a blood component from the centrifuge cartridge to a collection bag.

18. The apheresis system of claim 15, wherein at least one lumen conveys a blood component to a system for further processing of said blood component.

19. The apheresis system of claim 9, wherein the blood source is a patient.

20. The apheresis system of claim 9, wherein the blood source is a supply of blood already collected from a patient.

21. The apheresis system of claim 9, wherein the control system comprises a peristaltic block of independently controllable caterpillar rollers.

22. The apheresis system of claim 21, wherein the multi-lumen tube further includes a ribbon section, wherein the lumens are aligned in a plane so that each lumen may be positioned in contact with an independently controlled caterpillar roller of said peristaltic block.

23. The apheresis system of claim 22, wherein said ribbon section is bounded on each side by fittings of differing configurations.

24. The apheresis system of claim 23, wherein at least one of said fittings is asymmetric in form.

25. The apheresis system of claim 23, wherein the peristaltic block includes compatible recesses capable of receiving said ribbon section fittings.

26. The apheresis system of claim 9, wherein the top of the centrifuge cartridge is transparent.

27. The apheresis system of claim 26, further comprising a camera unit mounted above the centrifuge cartridge, wherein the camera unit is capable of differentiating a separation boundary within the centrifuge cartridge.

28. The apheresis system of claim 27, further comprising a microcontroller operable to receive separation boundary information from the camera unit, wherein said microcontroller is further capable of adjusting the angular velocity of the centrifuge cartridge based on said separation boundary information.

29. A method for separating whole blood obtained from a donor into at least two components, said method comprising:
a) supplying a source of whole blood to a sealless rotating drum, wherein the blood enters the drum via a dedicated lumen within a multi-lumen tube;
b) separating the whole blood into at least two components based on the density of said components via centrifugal forces placed on the components within a disposable rotating centrifuge cartridge located within the rotating drum; and
c) directing the blood components out of the rotating drum via separate dedicated lumens with the multi-lumen tube.

30. The method of claim 29, wherein the flow rate of blood and blood components within each lumen of the multi-lumen tube is independently variable.

31. The method of claim 29, wherein improved separation of blood components is achieved by increasing the angular velocity of the rotating centrifuge cartridge.

32. The method of claim 29, wherein improved separation of blood components is achieved by increasing the flow rate of an individual blood component.

33. The method of claim 29, wherein improved separation of blood components is achieved by decreasing the flow rate of an individual blood component.

34. The method of claim 29, wherein improved separation of blood components is achieved by increasing the flow rate of one blood component while simultaneously decreasing the flow rate of a different blood component.
